(19) United States
(12) Patent Application Publication
(10) Pub. No.: US 2005/0191340 A1
(43) Pub. Date: Sep. 1, 2005

(54) Opioid-receptor antagonists in transdermal systems having buprenorphine

(75) Inventors: Johannes Bartholomaeus, Aachen (DE); Heinrich Kugelmann, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/052,116

(22) Filed: Feb. 8, 2005

Related U.S. Application Data
(63) Continuation of application No. PCT/EP03/08828, filed on Aug. 8, 2003.

(30) Foreign Application Priority Data
Aug. 9, 2002 (DE) 102 37 056.7
Aug. 9, 2002 (DE) 102 37 057.5

(51) Int. Cl. A61K 31/485; A61K 9/70

(52) U.S. Cl. 424/449; 514/282

(57) ABSTRACT

Transdermal systems with an active agent such as buprenorphine and an opioid receptor antagonist are provided. The opioid receptor antagonist may include a μ, κ or δ opioid receptor antagonist. Methods of treatment using such a system are also provided.

### CROSS REFERENCE TO RELATED APPLICATIONS

This application is a continuation of International Patent Application No. PCT/EP2003/008828, filed Aug. 8, 2003, designating the United States of America, and published in German as WO 2004/014336 A2, the entire disclosure of which is incorporated herein by reference. Priority is claimed based on German Patent Application Nos. 102 37 056.7 and 102 37 057.5, both filed Aug. 9, 2002.

### FIELD OF THE INVENTION

The invention relates to opioid receptor antagonists, such as, for example naloxone, in transdermal forms of administration, having at least buprenorphine as an active ingredient and treatment methods involving the same.

### BACKGROUND

Products containing anaesthetising agents and, in particular, opioids basically run the risk of being abused, the level of this risk of abuse generally depending on the cost at which the potential abuser can achieve the desired effect of euphoria. Owing to the poor oral accessibility of most opioids, parenteral administration of an opioid-containing solution is usually a prerequisite for achieving such an effect of euphoria. In the event of abuse, attempts are made to obtain a parenterally administrable form of pharmaceutical composition, even though the medicine abused for this purpose is not available in a form which is primarily suitable for this. Abuse is obviously easiest with solutions of forms of pharmaceutical composition which can be completely dissolved. Properly produced parenteral solutions are clear, isotonic, isohydric, sterile and pyrogen-free and contain no undissolved constituents. It is virtually impossible to meet all these requirements during improper production; however, the accompanying health risk is taken into consideration. Transdermal systems, on which the invention focuses, are unsuitable for improper purposes, on account of the construction thereof. Nevertheless, a potential risk of abuse of opioid-containing patches, on account of the attractiveness of the active ingredient, basically cannot be ruled out. In this case, the anaesthetising agent would have to be dissolved from the matrix, and—if this can actually be achieved—the auxiliaries which are inevitably also eluted would further contaminate the solution, so a parenteral form of administration obtained in this way is unattractive.

Therefore, transdermal systems appear to be fundamentally unsuitable for abuse. Accordingly there have not hitherto been any further obstacles to abuse in transdermal systems in the prior art either. As a mere precaution, however, calculations should allow for the fact that abusers might potentially discover ways of overcoming the obstacles to abuse inherent in the transdermal system.

### SUMMARY OF THE INVENTION

One object of the invention is to provide a way of further reducing the risk of abuse of opioid-containing, in particular buprenorphine-containing, transdermal systems.

The invention accordingly relates to a transdermal system or transdermal therapeutic system containing as the active ingredient at least buprenorphine in the form of the base or of a salt, characterized in that it contains at least an μ, κ or δ opioid receptor antagonist; optionally in the form of its racemates, its pure stereoisomers, in particular enantiomers or diastereomers, or in the form of mixtures of the stereoisomers, in particular the enantiomers or diastereomers, in any mixing ratio; in the form of the acids or bases or in the form of the salts, in particular the physiologically acceptable salts, or in the form of the solvates, in particular the hydrates.

It has accordingly been found that this makes abuse more difficult to impossible. A parenterally acting opioid antagonist is simultaneously added to the transdermal system or the buprenorphine-containing patch is additionally coated with an opioid antagonist solution. Therefore, when attempting to dissolve the buprenorphine from the patch, the antagonist also has to be dissolved. During parenteral administration of such a solution, the antagonist immediately occupies the receptors, owing to its high affinity, for example, for the μ-opioid receptor, and thus prevents the effect of euphoria as receptor binding of the buprenorphine as the agonist is no longer possible.

The term “salt” generally denotes any form of an active ingredient according to the invention in which it assumes or is charged with an ionic form and is coupled to a counter ion (a cation or anion) or is in solution. This also includes complexes of the active ingredient with other molecules and ions, in particular complexes which are complexed by ion interaction.

The term “(physiologically acceptable) salt, in particular with acids” according to this invention denotes salts of at least one of the compounds according to the invention—usually protonated, for example on a nitrogen—as a cation with at least an anion which are physiologically acceptable—in particular when administered to humans and/or mammals. In the context of this invention it denotes, in particular, the salt formed with a physiologically acceptable acid, namely salts of the respective active ingredient with inorganic or organic acids, which are physiologically acceptable—in particular when administered to humans and/or mammals. Examples of physiologically acceptable salts of specific acids are salts of: hydrochloric acid, hydrobromic acid, sulphuric acid, methane sulphonic acid, formic acid, acetic acid, oxalic acid, succinic acid, tartaric acid, mandelic acid, fumaric acid, lactic acid, citric acid, glutamic acid, 1,1-dioxo-1,2, dihydro-1λ⁶-benzo[d]isothiazol-3-one (saccharic acid), monomethyl sebacic acid, 5-oxo-proline, hexane-1-sulphonic acid, nicotinic acid, 2-, 3- or 4-amino benzoic acid, 2,4,6-trimethyl-benzoic acid, α-lipoic acid, acetyl glycine, acetylsalicyclic acid, hippuric acid and/or aspartic acid. The hydrochloride, hydrobromide salt and the hydrogen citrate are particularly preferred.

It is particularly preferred if the μ, κ or δ opioid receptor antagonist contained in the transdermal system according to the invention is a μ opioid receptor antagonist or morphine antagonist, preferably levallorphan, naltrexone, nalorphine or naloxone, in particular naloxone.

This embodiment is particularly preferred if the levallorphan, naltrexone, nalorphine or naloxone contained in the pharmaceutical composition according to the invention is in the form of its base or in the form of the salts, in particular the physiologically acceptable salts, preferably the salts formed with inorganic or organic acids, in particular as the chloride-bromide salt or hydrogen citrate salt.

It is particularly preferred if the buprenorphine contained in the transdermal system according to the invention is in the form of a base.

According to Zaffaroni, the term “transdermal therapeutic system (TTS) or transdermal system” denotes “a device or a form of administration containing pharmaceutical substances which continuously delivers one or more pharmaceutical substances at a predetermined rate over a fixed period of time to a fixed site of administration” (quoted by Heilmann, “therapeutische Systeme-Konzept und Realisation programmierter Arzneiverabreichung”, 4th edition, Ferdinand Enke-Verlag Stuttgart 1984, page 26), the skin being the site of administration in the present case.

The construction of transdermal systems is known to a person skilled in the art. Intellectual property rights in which the fundamental construction is described include, for example, Patent Nos. DE 3315272, DE 3843239 and U.S. Pat. No. 3,598,122. The content of those patent specifications and applications is expressly incorporated herein by reference.

If a transdermal therapeutic system is applied to a patient's skin, the pharmaceutical substance should be delivered so as to act topically or systemically toward the patient. Pharmaceutical compositions in these forms are already used therapeutically. They are usually constructed in layers and, in the simplest case, consist of a backing layer, a self-adhesive active ingredient reservoir and a releasable protective layer that should be removed prior to administration: Transdermal systems of the type described in WO98/36728, WO 96/19975, U.S. Pat. No. 6,264,980, EP 430 019 A2 and CA 2030178 are particularly preferred, the disclosures of those specifications and the content of the literature cited therein are expressly incorporated herein by reference, including the nomenclature and definitions of the latter two documents. According to the invention at least a μ, κ or δ opioid antagonist is added to the transdermal systems.

The μ, κ or δ opioid antagonist may be added
a) by coating the surface of the transdermal system facing the skin with at least a μ, κ or δ opioid antagonist during therapeutic use,
b) by adding at least a μ, κ or δ opioid antagonist to the active ingredient,
c) by adding at least a μ, κ or δ opioid antagonist to the active ingredient reservoir layer or reservoir layer,
d) by coating the backing layer, which is impermeable to active ingredient, with at least a μ, κ or δ opioid antagonist, on the side facing the active ingredient reservoir layer or reservoir layer and/or
e) by coating the backing layer, which is impermeable to active ingredient, with at least a μ, κ or δ opioid antagonist, on the side remote from the active ingredient reservoir layer or reservoir layer.

The combination of buprenorphine base and naloxone salt is most preferred.

In a particularly preferred embodiment of the transdermal system according to the invention the ratio by weight of the amount of the μ, κ or δ opioid antagonists used in/on the pharmaceutical composition to the amount of μ, κ or δ opioid agonists used in the pharmaceutical composition is between 1:100 and 100:1, preferably between 1:20 and 10:1, in particular between 1:10 and 1:1, particularly preferably between 1:10 and 3:10.

In another preferred embodiment, providing that the transdermal system remains in contact with the skin for at least 5 days, it maintains an average release rate from about 3 μg/h to about 86 μg/h and an increase in the plasma level of the opioid agonist, in particular buprenorphine, basically of the first order from commencement of the dosing interval to about 72 hours after initiation of the dosing interval; and maintains an average release rate from about 0.3 μg/h to about 9 μg/h and an increase in the plasma level of the opioid agonist, in particular buprenorphine, basically of the zero-th order from about 72 hours after commencement of the dosing interval to the end of the at least 5-day dosing interval, so the following average plasma concentrations are achieved: an average plasma concentration from about 0.3 to about 113 pg/ml about 6 hours after commencement of the dosing interval;
an average plasma concentration from about 3 to about 226 pg/ml about 12 hours after commencement of the dosing interval;
an average plasma concentration from about 7 to about 644 pg/ml about 24 hours after commencement of the dosing interval;
an average plasma concentration from about 13 to about 753 pg/ml about 36 hours after commencement of the dosing interval;
an average plasma concentration from about 16 to about 984 pg/ml about 48 hours after commencement of the dosing interval;
an average plasma concentration from about 20 to about 984 pg/ml about 60 hours after commencement of the dosing interval;
an average plasma concentration from about 21 to about 1052 pg/ml about 72 hours after commencement of the dosing interval; and
an average plasma concentration from about 19 to about 1052 pg/ml for about 24 hours over at least the next 48 hours.

The invention further relates to the use of a transdermal system according to the invention for producing a pharmaceutical composition for the treatment of pain, in particular acute, chronic, visceral or neuropathic pain, or pain caused by inflammation, or for the treatment of an increased urge to urinate or urinary incontinence and related methods of treatment.

Abuse is made more difficult to impossible when the invention described here is carried out. Overall, a parenterally acting opioid antagonist is simultaneously added to the buprenorphine-containing patch or the opioid-containing patch is simultaneously additionally coated with an opioid antagonist-solution. Therefore, when attempting to dissolve the buprenorphine from the patch, the antagonist is also dissolved. During parenteral administration of such a solution, the antagonist immediately occupies the receptors, owing to its high affinity, for example for the it-opioid-receptor, and thus prevents the effect of euphoria, as receptor binding of the opioid as an agonist is no longer possible.

Any suitable μ receptor or μ and κ receptor antagonist such as, for example, nalorphine or naloxone in the form of its bases or salts with inorganic (for example, chloride) or organic (for example, hydrogen citrate) acids may be used as opioid antagonists. Preferably the opioid antagonist is used in the form of a compound that is substantially inaccessible transdermally but is readily soluble. The object of the invention is achieved in that the buprenorphine can act transdermally as an analgesic, but during the attempt to dissolve the buprenorphine out, the antagonist is also dissolved out and, during improper parenteral administration of the solution, prevents the buprenorphine from being effective.

The amount of opioid antagonist added is based on the total amount of opioid-agonist or buprenorphine contained in the product. The ratio of opioid agonist to buprenorphine is preferably 10:1 to 1:10.

### EXAMPLES

The following examples serve to illustrate certain embodiments of the present invention, but are not intended to and do not restrict the scope of protection thereof.

### Example 1

Incorporation of naloxone hydrochloride into the active ingredient matrix.

1125 g of a 48% by weight polyacrylate solution of a self-crosslinking acrylate copolymer of 2-ethylhexyl acrylate, vinyl acetate, acrylic acid (solvent: ethyl acetate:heptane:isopropanol:toluene:acetylacetonate in a ratio of 37:26:26:4:1), 100 g laevulinic acid, 150 g oleyl acetate, 100 g polyvinylpyrrolidone, 150 g ethanol, 200 g ethyl acetate, 100 g buprenorphine base and 20 g naloxone hydrochloride are homogenized. The mixture is stirred for approximately 2 hours and checked visually to ascertain whether all the solids have dissolved. The evaporation loss is checked by weighing, and the loss of solvent is optionally made up by ethyl acetate.

The mixture is then applied to a 420 mm wide transparent polyester sheet so that the weight per unit area of the dried layer of adhesive is 80 g/m². The protective layer is a silicone-treated releasable polyester sheet.

The solvent is removed by drying with heated air, which is conveyed over the moist web. The heat treatment causes the solvents to evaporate. The adhesive film is then covered with a 15 μm polyester sheet. An area corresponding to the proposed amount of active ingredient is punched out using suitable cutting tools and the borders remaining between the individual systems are removed.

An exemplary method for producing such a patch is provided in Example 1 of WO 96/19975 and EP 430 019 A2.

### Example 2

Coating of the active ingredient matrix with naloxone hydrochloride.

1139 g of a 48% by weight polyacrylate solution of a self-crosslinking acrylate copolymer of 2-ethylhexyl acrylate, vinyl acetate, acrylic acid (solvent: ethyl acetate:heptane:isopropanol:toluene:acetylacetonate in a ratio of 37:26:26:4:1), 100 g laevulinic acid, 150 g oleyl acetate, 100 g polyvinylpyrrolidone, 150 g ethanol, 200 g ethyl acetate and 100 g buprenorphine base are homogenized. The mixture is stirred for approximately 2 hours and checked visually to ascertain whether all the solids have dissolved. The evaporation loss is checked by weighing, and the loss of solvent is optionally made up by ethyl acetate.

The mixture is then applied to a 420 mm wide transparent polyester sheet so that the weight per unit area of the dried layer of adhesive is 80 g/m². The protective layer is a silicone-treated releasable polyester sheet.

The solvent is removed by drying with heated air, which is conveyed over the moist web. The heat treatment causes the solvents to evaporate. The matrix is then coated with a solution of 800 mg naloxone hydrochloride in 120 ml methanol per m² by homogeneous spraying, sprinkling or trickling, and the solvent removed by drying again.

The adhesive film is then covered with a 15 μm polyester sheet. An area corresponding to the proposed amount of active ingredient is punched out using suitable cutting tools and the borders remaining between the individual systems are removed.

An exemplary method for producing such a patch is provided in Example 1 of WO 96/19975 and EP 430 019 A2.

### Example 3

Coating of the polyester sheet with naloxone hydrochloride on the side facing the active ingredient matrix.

1139 g of a 48% by weight polyacrylate solution of a self-crosslinking acrylate copolymer of 2-ethylhexyl acrylate, vinyl acetate, acrylic acid (solvent: ethyl acetate:heptane:isopropanol:toluene:acetylacetonate in a ratio of 37:26:26:4:1), 100 g laevulinic acid, 150 g oleyl acetate, 100 g polyvinylpyrrolidone, 150 g ethanol, 200 g ethyl acetate and 100 g buprenorphine base are homogenized. The mixture is stirred for approximately 2 hours and checked visually to ascertain whether all the solids have dissolved. The evaporation loss is checked by weighing, and the loss of solvent is optionally made up by ethyl acetate.

A 420 mm wide transparent polyester sheet is coated with a solution of 800 mg naloxone hydrochloride in 120 ml methanol per m², and the solvent is removed by drying. The above-described mixture is then applied to the coated side of the coated polyester sheet so that the weight per unit area of the dried adhesive layer is 80 g/m 2. The protective layer is a silicone-treated releasable polyester sheet.

The solvent is removed by drying with heated air, which is conveyed over the moist web. The heat treatment causes the solvents to evaporate.

The adhesive film is then covered with a 15 μm thick polyester sheet. An area corresponding to the proposed amount of active ingredient is punched out using suitable cutting tools and the borders remaining between the individual systems are removed.

An exemplary method for producing such a patch is provided in Example 1 of WO 96/19975 and EP 430 019 A2.

### Example 4

Coating of the polyester sheet with naloxone hydrochloride on the side remote from the active ingredient matrix.

1139 g of a 48% by weight polyacrylate solution of a self-crosslinking acrylate copolymer of 2-ethylhexyl acrylate, vinyl acetate, acrylic acid (solvent: ethyl acetate:heptane:isopropanol:toluene:acetylacetonate in a ratio of 37:26:26:4:1), 100 g laevulinic acid, 150 g oleyl acetate, 100 g polyvinylpyrrolidone, 150 g ethanol, 200 g ethyl acetate and 100 g buprenorphine base are homogenized. The mixture is stirred for approximately 2 hours and checked visually to ascertain whether all the solids have dissolved. The evaporation loss is checked by weighing, and the loss of solvent is optionally made up by ethyl acetate.

A 420 mm wide transparent polyester sheet is coated with a solution of 800 mg naloxone hydrochloride in 120 ml methanol per m², and the solvent is removed by drying. The sheet is then turned over so that the uncoated side is directed upwards. The above-described mixture is then applied to the uncoated side of the coated polyester sheet so that the weight per unit area of the dried adhesive layer is 80 g/m². The protective layer is a silicone-treated releasable polyester sheet.

The solvent is removed by drying with heated air, which is conveyed over the moist web. The heat treatment causes the solvents to evaporate.

The adhesive film is then covered with a 15 μm thick polyester sheet. An area corresponding to the proposed amount of active ingredient is punched out using suitable cutting tools and the borders remaining between the individual systems are removed.

An exemplary method for producing such a patch is provided in Example 1 of WO 96/19975, U.S. Pat. No. 6,246,980, EP 430 019 A2 and CA 2030178.

### Example 5

Permeation tests through stratum corneum.

Permeation of naloxone HCl in addition to buprenorphine (base) from a transdermal system produced as in Examples 1 to 4.

1. Construction of Penetration Model

Franz cell with an acceptor volume of about 100 ml. A suitable magnetic fish is first introduced into the acceptor chamber. The contact faces for fixing the permeation barrier (stratum corneum here) are brushed with silicone paste (lubricant), and a polycarbonate filter with a pore width of 5 μm is applied centrally on the acceptor side (Millipore TMTP025). The punched-out undamaged piece of stratum corneum, which is hydrated prior to use, is placed on this support membrane and the piece of patch to be tested is applied thereto as a donor. The upper part of the Franz cell is then applied and fixed to the lower part by a clip.

The degassed acceptor medium, which is about 34° C., is introduced without bubbles via the riser pipe. The amount of acceptor filling is selected so as to ensure complete contact between the acceptor and the permeation barrier. The liquid level in the riser pipe is invariably above the plane of the permeation barrier.

To weight the donor-side patch, an HPLC vial is placed on the surface thereof and the donor-side nozzle sealed with parafilm or another sealing system.

At the beginning of the test, the prepared Franz cell is suspended in a water bath, which is thermostatically controlled to 34° C., in such a way that the bath liquid virtually completely reaches the acceptor medium, but there is no risk of it passing into the Franz cell at the interface between donor region and acceptor region.

The bath with the Franz cell is placed on a magnetic stirrer in such a way that both bath liquid and acceptor medium are stirred via magnetic fishes.

2. Donor

The cut-to-size patch acts as the donor.

3. Acceptor

An isotonic phosphate buffer solution containing the following salts dissolved in a 1 l aqueous solution serves as the acceptor:
1.225 g potassium dihydrogen phosphate, anhydrous
4.26 g disodium hydrogen phosphate, anhydrous
6.00 g sodium chloride
Sampling for analytical purposes: about 10 ml
   4. Permeation Barrier

The stratum corneum (s. c.) acts as the permeation barrier.

Storage with drying agent in the desiccator at ambient temperature since 9.4.2002.

Prior to use, the s. c. is examined under a microscope and corresponding areas are selected as a barrier and cut out.

5. Sampling

Sampled Amounts

2×200 μl are removed using a varipette at each sampling instant and the respectively removed amount is replaced by isotonic phosphate buffer solution.

Sampling with the varipette takes place at each sampling instant, and a new pipette tip is used for each addition of buffer solution.

The varipette is adjusted to a value which is required for delivering an average of 200 μl (203 μl).

All removed samples are additionally weighed and documented. The removed samples are placed in HPLC vials, sealed, labelled and frozen.

6. Sampling Times:

2 samples of 200 μl are taken from each series after 0.5; 1.0; 1.5; 2; 4; 6; 24; and 48 hours respectively.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A transdermal therapeutic system comprising:
buprenorphine or a physiologically acceptable salt thereof and
a μ, κ or δ opioid receptor antagonist or a physiologically acceptable salt thereof.

2. The transdermal therapeutic system of claim 1, wherein said buprenorphine is in the form of a base.

3. The transdermal therapeutic system of claim 1, wherein said opioid receptor antagonist is present in the form of a pure enantiomer or pure diastereoisomer.

4. The transdermal therapeutic system of claim 1, wherein said opioid receptor antagonist is present in the form of a mixture of stereoisomers.

5. The transdermal therapeutic system of claim 1, wherein said opioid receptor antagonist is present in the form of a racemic mixture.

6. The transdermal therapeutic system of claim 1, wherein said opioid receptor antagonist is present in the form of a base.

7. The transdermal therapeutic system of claim 1, wherein said opioid receptor antagonist is present in the form of an acid.

8. The transdermal therapeutic system of claim 1, wherein said opioid receptor antagonist is present in the form of a solvate.

9. The transdermal therapeutic system of claim 1, wherein said opioid receptor antagonist is present in the form of a hydrate.

10. The transdermal therapeutic system of claim 1, wherein the opioid receptor antagonist is a μ opioid receptor antagonist or morphine antagonist.

11. The transdermal therapeutic system of claim 1, wherein the opioid receptor antagonist is selected from the group consisting of levallorphan, naltrexone, nalorphine or naloxone.

12. The transdermal therapeutic system of claim 11, wherein the opioid receptor antagonist is present in the form of a chloride-bromide salt or a hydrogen citrate salt.

13. The transdermal therapeutic system of claim 1, wherein the opioid receptor antagonist is naloxone.

14. The transdermal therapeutic system of claim 1, wherein the μ, κ or δ opioid receptor antagonist is provided
a) by coating the surface of the transdermal system facing the skin with at least a μ, κ or δ opioid antagonist;
b) by incorporating at least a μ, κ or δ opioid antagonist in the active ingredient;
c) by adding at least a μ, κ or δ opioid antagonist to an active ingredient reservoir layer or reservoir layer;
d) by coating a backing layer, which is impermeable to the active ingredient, with at least a μ, κ or δ opioid antagonist, on a side facing an active ingredient reservoir layer or reservoir layer or
e) by coating a backing layer, which is impermeable to the active ingredient, with at least a μ, κ or δ opioid antagonist, on a side remote from an active ingredient reservoir layer or reservoir layer.

15. The transdermal therapeutic system of claim 1, wherein the ratio by weight of the amount of μ, κ or δ opioid receptor antagonist used in the pharmaceutical composition to the amount of buprenorphine is between 1:100 and 10:1.

16. The transdermal therapeutic system of claim 1, wherein the ratio by weight of the amount of μ, κ or δ opioid receptor antagonist used in the pharmaceutical composition to the amount of buprenorphine is between 1:20 and 5:1.

17. The transdermal therapeutic system of claim 1, wherein the ratio by weight of the amount of μ, κ or δ opioid receptor antagonist used in the pharmaceutical composition to the amount of buprenorphine is between 1:10 and 1:1.

18. The transdermal therapeutic system of claim 1, wherein the ratio by weight of the amount of μ, κ or δ opioid receptor antagonist used in the pharmaceutical composition to the amount of buprenorphine is between 1:10 and 3:10.

19. The transdermal therapeutic system of claim 1, wherein, provided that the transdermal therapeutic system remains in contact with skin for at least 5 days, the transdermal therapeutic system maintains an average release rate of from about 3 μg/h to about 86 μg/h and an increase in the plasma level of the buprenorphine, basically of the first order from commencement of the dosing interval to about 72 hours after initiation of the dosing interval; and maintains an average release rate from about 0.3 μg/h to about 9 μg/h and an increase in the plasma level of the opioid agonist, in particular buprenorphine, basically of the zero order from about 72 hours after commencement of the dosing interval to the end of the at least 5-day dosing interval, so the following average plasma concentrations are achieved:
an average plasma concentration from about 0.3 to about 113 pg/ml about 6 hours after commencement of the dosing interval;
an average plasma concentration from about 3 to about 226 pg/ml about 12 hours after commencement of the dosing interval;
an average plasma concentration from about 7 to about 644 pg/ml about 24 hours after commencement of the dosing interval;
an average plasma concentration from about 13 to about 753 pg/ml about 36 hours after commencement of the dosing interval;
an average plasma concentration from about 16 to about 984 pg/ml about 48 hours after commencement of the dosing interval;
an average plasma concentration from about 20 to about 984 pg/ml about 60 hours after commencement of the dosing interval;
an average plasma concentration from about 21 to about 1052 pg/ml about 72 hours after commencement of the dosing interval; and
an average plasma concentration from about 19 to about 1052 pg/ml for about 24 hours over at least the next 48 hours.

20. A method of alleviating pain or treating an increased urge to urinate or urinary incontinence in a mammal, said method comprising administering to said mammal a transdermal therapeutic system according to claim 1.

21. The method of claim 20, wherein said pain is acute, chronic, visceral or neuropathic pain or pain caused by inflammation.
